(19) United States
(12) Patent Application Publication
(10) Pub. No.: US 2026/0346008 A1
(43) Pub. Date: Nov. 5, 2026

(54) ISOLATING MOUTHGUARD FOR ELECTRICITY-BASED THERAPIES

(71) Applicant: Cur Therapeutics, Inc., Kirkland, WA (US)

(72) Inventors: Whitney Jackson, Kirkland, WA (US); Harrison Farrell, Tampa, FL (US)

(73) Assignee: Cur Therapeutics, Inc., Kirkland, WA (US)

(21) Appl. No.: 19/660,377

(22) Filed: Apr. 28, 2026

Related U.S. Application Data
(60) Provisional application No. 63/800,088, filed on May 5, 2025.

(51) Int. Cl. A61N 1/36; A61N 1/32

(52) U.S. Cl. CPC A61N 1/36034; A61N 1/328

(57) ABSTRACT

Aspects relating to a method and system for isolating an electrically conductive material within a subject from a current administered as part of an electricity-based therapy. The current may be a microcurrent. The method and system including inserting an insulating device, such as a mouthguard into the mouth of a subject to isolate or otherwise minimize the transmission of an electrical current to electrically conductive material from the current generating device exterior to a subject’s skin, said using said device to administer a controlled electric current to the skin of the subject’s face.

### RELATED APPLICATIONS

This Application is a Non-Provisional of U.S. Application Serial No. 63/800,088, filed May 5, 2025, entitled "ISOLATING MOUTHGUARD FOR ELECTRICITY-BASED THERAPIES", the entire contents of which is incorporated herein by reference in its entirety.

### BACKGROUND

### 1. Field of Invention

This invention relates to systems and methods of using electricity-based therapies with a device that shields conductive implants or other electrically conductive materials from the generated current.

### 2. Related Art

The use of electrical current to treat skin and muscle layers have been implemented in the art. Such electrical currents may be generated for pulsed electromagnetic field stimulation, interferential therapy, frequency specific microcurrents, microcurrent electrical nerve stimulation, and/or transcutaneous electrical nerve stimulation.

### SUMMARY OF INVENTION

Aspects of the disclosure relate to a method of using a mouthguard with an electricity-based therapy. The method may include positioning the mouthguard at least partially over an electrically conductive material on or near a subject’s teeth so that the mouthguard is positioned between the electrically conductive material and a target region of the subject (e.g. an area that is targeted with the administration of an electricity-based therapy). The electricity-based therapy may be administered to the target region, and during such administration, the electrically conductive material may be isolated from the electricity-based therapy by the mouthguard. The mouthguard may isolate the electrically conductive material by preventing, reducing, limiting or otherwise controlling a transmission of a current generated by the electricity-based therapy through the skin of a subject to the electrically conductive material.

The shape of the mouthguard may help control the transmission of a current from a current generating device to an electrically conductive material. For example, the mouthguard may have included a front wall that covers the anterior side of a subject’s teeth, a rear wall that contacts the posterior side of the subject's teeth and/or a base that connects the front wall and the rear wall. In some embodiments, the rear wall may include a tongue portion that may separate the tongue from the covered teeth. Depending on the orientation of the mouthguard in a subject’s mouth, the tongue portion may contact the sublingual space or the hard palate of a subject.

In some embodiments, the composition and structure of the mouthguard may control the transmission of the current to the electrically conductive material. For example, the mouthguard may be fabricated from a material that has low or reduced conductivity, e.g., is an insulator. As such, a material may isolate the electrically conductive material by limiting or otherwise controlling the transmission of the electrical current from the skin to the electrically conductive material. Therefore, the ability of the mouthguard to electrically isolate the electrically conductive material may be adjusted based on the mouthguard’s composition and/or construction.

The mouthguard may be partly, primarily, or fully fabricated from electrically insulating material such as plastics including silicone, rubber, ethylene-vinyl acetate, and/or polyvinyl chloride. However, this list may not be exhaustive as other electrically insulating materials or materials otherwise having a reduced or low conductivity, such as a ceramic, may be used to isolate the electrically conductive material.

In some cases, the thickness of the mouthguard at one or more areas may be determined based on the desired isolation. In some cases, a thicker material section may be more capable of limiting or otherwise controlling the flow of an electrical current. As such, the isolating properties of the mouthguard may be altered by changing the thickness of the mouthguard in one or more portions. In some embodiments, the thickness of the front wall, base and/or the rear wall may be consistent throughout the mouthguard. Alternatively, or additionally, the thickness of the front wall, the base and/or the rear wall may be variable, e.g., the front wall may be thicker in one or more portions than the rear wall. In some cases, different regions of a mouthguard may vary in thickness, such as different regions of the front wall. For example, a central area associated with a front portion of the front wall that is configured to be placed proximal to a subject’s incisors may be thicker than an outer portion of the front wall intended to be placed proximal to a subject’s more rearward teeth. Accordingly, one or more portions of the mouthguard may be made thicker or thinner relative to each other based on the desired electrically isolating properties of different regions of the mouthguard.

In some embodiments a system for administering an electricity-based therapy may include a device configured to administer the electricity-based therapy to a target region of a subject and a mouthguard. The mouthguard may be designed to electrically isolate electrically conductive material within a subject’s mouth from the electricity-based therapy by controlling a transmission of a current generated by the electricity-based therapy along and/or through the mouthguard.

In some embodiments, it may be desirable for the mouthguard to be flexible and electrically insulative, and the material and thickness of the mouthguard may also be selected based on the desired flexibility of the mouthguard. Accordingly, in some embodiments, a front wall of the mouthguard may be bendable to fit within the mouth of a subject which may allow the mouthguard to deform so that it can accommodate for the different sizes and/or shapes between different subjects’ mouths.

These and other aspects of the invention will be apparent from the following description and claims.

### BRIEF DESCRIPTION OF DRAWINGS

Aspects of the invention are described below with reference to the following drawings in which like numerals reference like elements, and wherein:

FIG. 1 is an illustration of a subject receiving an electricity-based therapy;

FIG. 2 shows a flow chart of the method of using a mouthguard with the electricity-based therapy;

FIG. 3 shows a perspective view of a mouthguard according to one embodiment;

FIG. 4 shows a second perspective view of the mouthguard detailed in FIG. 3; and

FIG. 5 shows a perspective view of a mouthguard according to a second embodiment.

### DETAILED DESCRIPTION

It should be understood that aspects of the disclosure are described herein with reference to certain illustrative embodiments and the figures. The illustrative embodiments described herein are not necessarily intended to show all aspects of the disclosure but rather are used to describe a few illustrative embodiments. Thus, aspects of the disclosure are not intended to be construed narrowly in view of the illustrative embodiments. In addition, it should be understood that aspects of the disclosure may be used alone or in any suitable combination with other aspects of the invention.

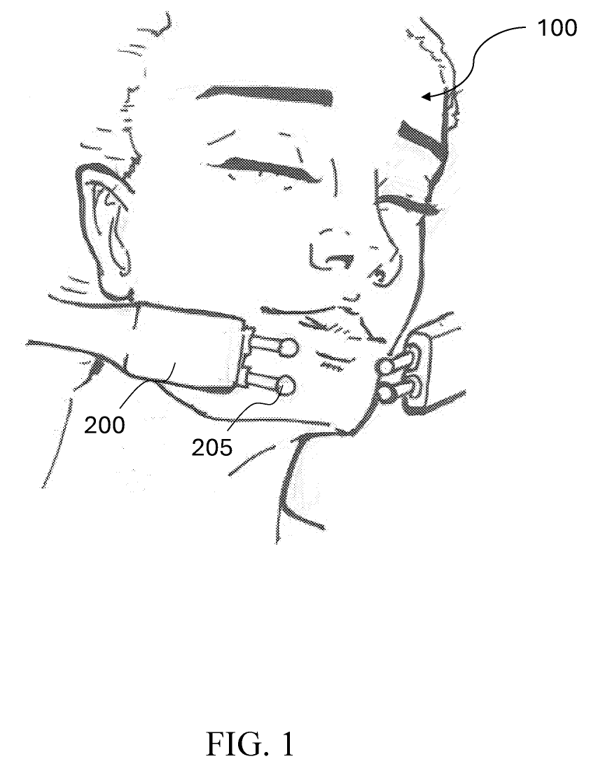

Electricity-based therapies may include the application of a current to a specific region of a patient or a subject. These therapies may take various forms but are typically administered to a targeted general region where the benefits are sought, such as the skin of the subject. FIG. 1 illustrates a current generating device 200 capable of administering an electricity-based therapy to a subject 100. Such devices and therapies may include those disclosed in U.S. Pat. No. 10,463,531; U.S. Pat. No. 6,408,211; U.S. Pat. No. 4,989,605; U.S. Pat. No. 8,998,791; and U.S. Pat. No. 9,155,890. These devices generally include at least one electrode 205 that may include an electrical source and that may direct the flow of a current to the target region on the subject. In some instances, the target region is in contact with the electrode, or in some instances, such as when the target area is a layer of muscle, the electrode is not in physical contact with the target region but is capable of generating a current that may reach or otherwise flow at the target area. As such, the electrode may be, or not be, in electrical contact with the target area where current flow through a subject is generated.

In some embodiments, the current generating device may include a cathode and an anode, which may flow a current through a subject from the positive pole to the negative pole. It should be appreciated that the flow of current through the body of a subject causes a flow of negatively charged particles from the anode to the cathode. Furthermore, the cathode may generate a negative pole that attracts positively charged ions within the tissue, while the anode may generate a positive pole that attracts negatively charged ions, which creates a bidirectional ionic current. The positive hydrogen ions at the negatively charged cathode, may increase ATP production in the targeted region, increasing the available energy for a subject’s cells.

As noted above, electricity-based therapies may include a wide range of therapies. For example, in some instances an electricity-based therapy may be a cosmetic procedure such as, but not limited to, electromagnetic facials, microcurrent facials, or an electrical muscle stimulation. These therapies may target the skin to tone and lift and may also have clinical applications, such as the treatment of Bell’s Palsy.

In some embodiments, the device may generate a single current, such as frequency-specific microcurrent devices. Alternatively, or additionally the device may be used to administer multiple electrical currents, which may be at slightly different frequencies. For example, the device may be an interferential current device that uses multiple electrodes to discharge multiple electrical impulses at different frequencies to generate a current within a target area that may offer pain relief or be part of a clinical treatment. The clinical treatments may include (but are not limited to) patients with arthritis, temporomandibular joint pain, and musculoskeletal pain. However other devices that use other electricity-based therapies such as, but not limited to, transcutaneous electrical nerve stimulation, microcurrent electrical neuromuscular stimulation may be substituted.

It should be appreciated that in some instances, the current may travel beyond the target region, into other portions of the body. In some instances, a subject may have an electrically conductive material within their body near the target region, which may cause discomfort within the subject during the above-mentioned therapies, and it may not be feasible or otherwise desirable to remove the electrically conductive materials. Moreover, the presence of these materials may impact the effectiveness of the treatment, as the electrically conductive material may act as an unanticipated grounding element altering the flow of current and/or ions in the tissue. As such, the inventors have recognized that it may be beneficial to electrically isolate the conductive material from the generated current during an electricity-based therapy with a shielding device. In some embodiments, the shielding device may electrically isolate the electrically conductive material without being electrically connected to the electricity-based therapy device, e.g., without being electrically connected to any grounding component of the therapy device. Thus, in some embodiments, the shielding device may not by grounded or may not otherwise be electrically connected to the current generating device in any way. Alternatively, or additionally, the shielding device may electrically isolate the electrically conductive material by otherwise discouraging the transmission of a current from the current-generating device into a region associated with the shielding device, such as the subject’s oral cavity. As will be appreciated by this disclosure, the shielding device may electrically isolate the conductive materials by minimizing or otherwise controlling the transmission of electrical currents through the shielding device or otherwise preventing or otherwise controlling electrical current from being transmitted to an electrically conductive material.

In some embodiments, the electrically conductive material may be within the mouth of the subject, and the target region may be along a subject’s face, or more specifically regions that are proximal to the mouth or jaw. The electrically conductive material may be removable or non-removable. In some embodiments, non-removable may mean that the material is permanent, or not intended to be removed, such as implants in the teeth or jaw of a subject. Alternatively, or additionally, non-removable material may mean that the device cannot be easily removed and reattached, for example certain piercings, or the electrically conductive material may require special tools or skills to remove or reattach, for example braces. In some embodiments, the non-removable material may be a non-removable dental device such as a dental implant, crown, filling and other orthodontics such as permanent retainers and braces. As such, in some embodiments, the shielding device may be designed to be placed within the mouth of a subject, for example the shielding device may be a mouthguard. However, the shielding device may be placed along different regions of the body, and as such, may have different forms to perform its isolating function.

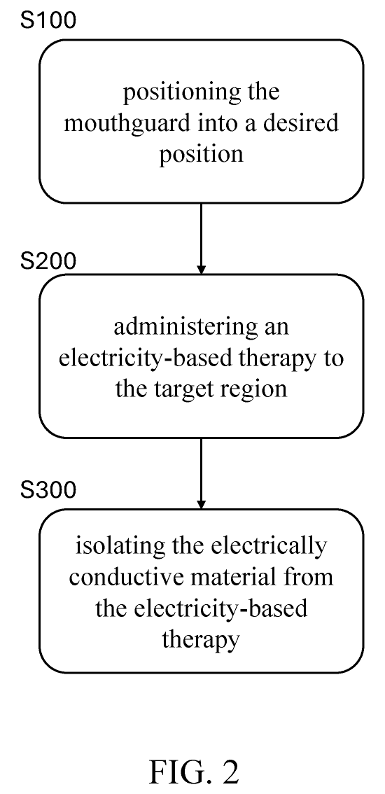

A method of controlling the transmission of a current from a current generating device that administers an electricity-based therapy may include use of a shielding device that is a mouthguard. As can be seen in FIG. 2, a first step S100 of the method may include positioning the mouthguard into a desired position. For example, this may include placing the mouthguard at least partially over an orthodontic or other dental device that includes an electrically conductive material on or near a subject’s teeth. In this position the mouthguard may be between the electrically conductive material and a target region of the subject.

A second step S200 may include administering an electricity-based therapy to the target region. In some embodiments, the target region may be associated with a subject’s face, such as the skin of the subject’s face. The frequency and power of the current used in the electricity-based therapy may vary depending on the application, for example a microcurrent therapy may include administering a current at a range of 1 microamperes to 999 microamperes and at a frequency between 0.5 Hz and 100 Hz. These ranges may be at a sub-sensory level for electrical currents, as such, the currents may be applied at a level that is below or similar to the currents generated by the subject’s nervous system. However, this may not be limiting as, the device may be used with different electricity-based therapies with different currents and/or different frequencies.

A third step S300 may include isolating the electrically conductive material from the electricity-based therapy with the mouthguard. The mouthguard may isolate the electrically conductive material by limiting or otherwise controlling the transmission of a current generated by the electricity-based therapy with respect to flow from the buccal mucosa to the teeth. In some embodiments, the flow of current may be interrupted by the composition of the mouthguard, as such the mouthguard may be fabricated from materials that are not electrically conductive and may be fabricated from electrically insulating materials. As such, once the current passes through the layers of the skin and muscle associated with the mouth region and passed into the oral cavity through the buccal and labial mucosa (i.e. the inside of a subject’s cheek and lips), the mouthguard may operate to further limit or otherwise control the flow of current.

In some embodiments, the electrically insulating materials may normally be in contact with the buccal and labial mucosa. In some cases, the buccal mucosa may contact the mouthguard. As such, this may enable the mouthguard to prevent the transmission of a current regardless of the location of the electrically conductive material along the dental arch. Therefore, it should be appreciated that the mouthguard may generally serve to minimize or otherwise control the transmission of the current along and through the mouthguard to the electrically conductive material.

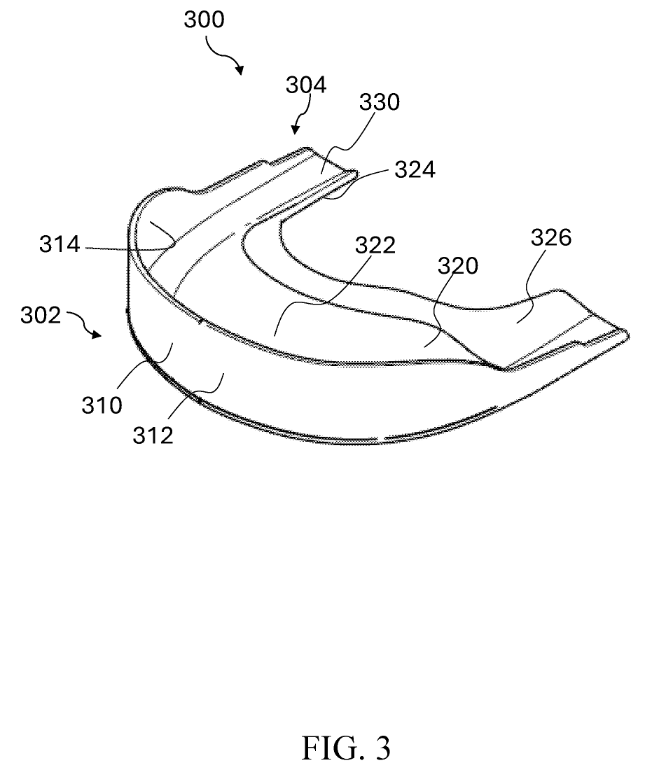

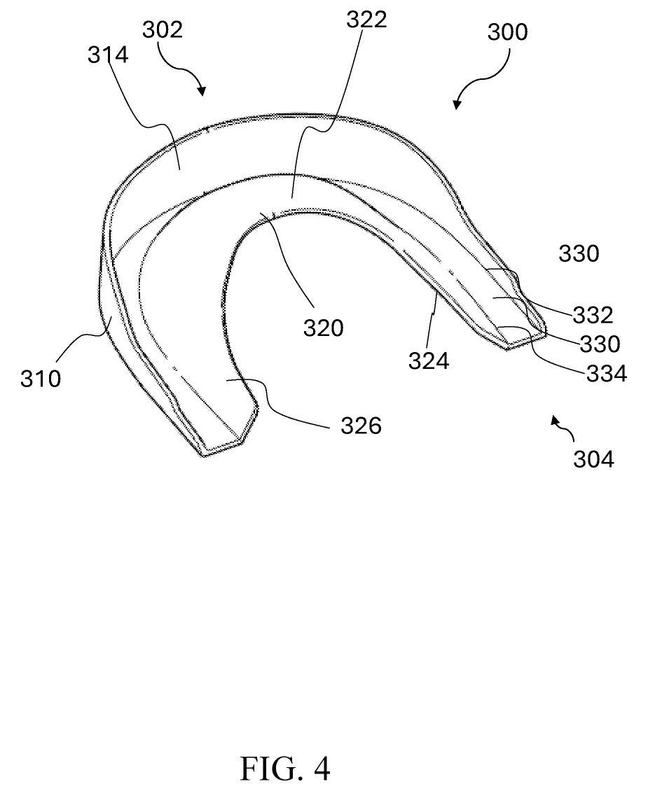

A mouthguard that may be used to limit the transmission of a current from a current generating device to an electrically conductive material is illustrated in FIGS. 3-4. The mouthguard 300 may have a front wall 310, a base 330 and a rear wall 320. The mouthguard 300 may have a general horseshoe or “u” shape or any shape that allows it to follow the general shape of a subject’s dental arch and to fit within their oral cavity. This geometry may physically separate and isolate a subject’s teeth from other anatomical structures.

The front wall 310 of the mouthguard may contact the buccal mucosa at a first exterior side 312 of the front wall, and a first interior wall 314 may contact the facial (or exterior) side of a subject’s teeth. This front wall 310 may be thick enough or otherwise configured to limit or otherwise control the transmission of a current to the teeth that are in contact with the first interior wall 314. Moreover, the front wall 310 may be thin enough for the mouthguard to cover at least a portion, the majority or the entire buccal and/or labial mucosa. As such, in some embodiments, a subject may be able to close their mouth so that the upper lip is in contact with their lower lip while using the mouthguard. This may enable the subject to receive the electricity-based therapy while the target region is positioned on or near the mouthguard.

In some cases, the front wall 310 may vary in height. For example, a portion of the front wall 310 near the front portion 302 of the mouthguard may be associated with the front teeth of a subject, and may be larger than another portion of the front wall near the outer portion 304 of the mouthguard that may be in contact with the molars of a subject. Accordingly, in some embodiments, the mouthguard 300 may cover portions of a subject’s gingiva or gums in addition to their teeth. For example, the guard may be sized to fit into a mucobuccal fold, or proximal to where buccal mucosa contacts the maxilla or the mandible.

Alternatively, or additionally, the mouthguard 300 may only cover a portion of the exterior side of a subject’s teeth so that portions may be exposed. For example, in embodiments where the current generating device administers electricity-based therapies that flow a very small current into the skin, the current may be partially or fully isolated from the molars within the oral cavity via adipose depots within fat pads (such as the buccal fat pad) within the subject’s cheek. Without wishing to be bound by any theory, because fat is a poor conductor of electricity, areas with large adipose deposits may not require as much insulating material as regions with less or different tissue. This shape may also promote user comfort, by ensuring that the end portion of the device easily fits within or conforms to the posterior region of a subject’s mouth. However, it should be noted that, in some instances, such as when a higher current is applied to the region, when the fat pads fail to isolate the electrically conductive material, or both, the insulating material of the mouthguard may be applied to the area to minimize discomfort.

The mouthguard may also contain a rear wall 320 on the opposite posterior side of the subject’s teeth relative to the front wall 310. This rear wall 320 may contact portions of the lingual side of the teeth and, as such, the rear wall may have a general arch shape where it connects to the base that follows the rear contour of a dental arch of a subject. Furthermore, the rear wall 320 may extend into a tongue portion 322 that contacts portions of the oral cavity that is posterior to a subject’s teeth, and as such may also be formed with a general arch shape from the base that follows the general contours of this region. Moreover, the posterior side of the tongue portion 322 may have an arch that mirrors the arch formed between the connection of the base to the rear wall. This tongue arch may aid in user comfort and may help seal the arch to the inside of the mouth.

This rear wall 320 may further isolate the electrically conductive material. However, in instances where the current is administered to a target region that is between the subject’s teeth and the rear wall 320, the rear wall may have no or minimal impact in isolating the electrically conductive material from the electricity-based therapy. In said application, the rear wall 320 may provide support for the subject to properly position the mouthguard in their mouth.

However, in some applications, the rear wall 320 may isolate the electrically conductive material. For example, the rear wall may include a tongue portion 322 which extends out from the rear wall to contact a subject’s palate when the mouthguard 300 is applied to the maxillary teeth (on a subject’s upper jaw). This contact may be used to isolate the electrically conductive material from the current transferred through the labial and buccal mucosa into a subject’s saliva. Generally, the mouth of a subject may be filled with saliva, which may act as a conduit for electricity. As such, a flat portion of the tongue portion 322 may form a seal between the mouthguard and a palate of the subject’s mouth to prevent the conduction of electricity from the oral cavity into the mouthguard and to the electrically conductive material via the subject’s saliva.

Although the previous embodiment states that the mouthguard 300 may be positioned on the maxillary teeth, in some embodiments, the mouthguard may be designed to be positioned on the mandibular teeth (associated with the lower jaw). Alternatively, or additionally, the mouthguard may be designed to be agnostic to its placement, and as such may be positioned on either the mandibular teeth or the maxillary teeth.

In some instances, it may be desirable to position the mouthguard on one jaw rather than the other, which may be influenced by the location of the electrically conductive material. For example, if a subject has a dental implant within their mandible or mandibular teeth, then the mouthguard 300 may be positioned over their lower jaw. Moreover, the electrically conductive material may not be associated with a subject’s teeth and instead may be associated with another part of the oral cavity, such as a piercing within the tongue. Accordingly, the tongue may need to be isolated from the current flowing from the current generating device through the mucosa into the oral cavity. As such, a tongue portion 322 of the mouthguard 300 may contact the subject’s tongue to isolate it from the current. The rear wall 320 with the tongue portion 322 may include a second interior wall 326, which contacts the lingual side of the teeth and the sublingual space under the tongue and a second exterior wall 324 that may contact a subject’s tongue. As suggested in the above embodiment, the flat portion of the tongue portion 322 may form a seal between the sublingual space and the mouthguard 300 to isolate the teeth positioned within the mouthguard 300. However, in this configuration, the tongue portion 322 may also isolate portions of the tongue from the sublingual salivary gland. Therefore, because a subject may rest their tongue against the electrically insulating material, and the tongue may be spaced from the subject’s saliva, the piercing may be isolated from the electrical current used in electricity-based therapies.

In addition to isolating the tongue and jaw, the mouthguard 300 may also isolate the maxillary teeth from the mandibular teeth via a base 330. This base 330 may connect the front wall 310 of the mouthguard from the rear wall 320 of the mouthguard. Naturally, the base may be U-shaped or otherwise follow the general dental arch of a subject’s teeth. In some embodiments, the base 330 may be designed to contact all of a subject’s teeth or otherwise be positioned between all of the maxillary teeth and their associated mandibular teeth. However, in some embodiments, the base 330 may be configured to be in contact with or be positioned between only a portion of the of the maxillary teeth and their associated mandibular teeth. In such an embodiment, the base 330 of the mouthguard 300 may be sufficiently thick to prevent the maxillary teeth from contacting the mandibular teeth. Moreover, in some embodiments, even when the mouthguard is within the subject’s mouth, the maxillary teeth may contact the mandibular teeth. As should be appreciated by the above disclosure, such an embodiment may be advantageous when a low current is applied to the facial region.

The distance between the front wall 310 and the rear wall 320 of the mouthguard may be determined by the base 330. The base 330 may maintain a consistent distance between its inner arch 334 and outer arch 332, which may cause the distance between the front wall 310 and the rear wall 320 to be constant throughout the mouthguard 300. Alternatively, the distance between the inner 334 and outer arch 332 of the base 330 may be variable, which may cause the distance between the front wall 310 and the rear wall 320 to change. This change may be based on the general anatomy of the teeth. For example, the incisors at the front of the mouth are typically narrower than the molars at the rear of the mouth, as such the molars require more space than the incisors. Accordingly, the distance between the outer arch 332 and the inner arch 334 of the base may be smaller at the front portion 302 of the mouthguard and larger at the outer portion 304 of the mouthguard.

In some embodiments, the base 330 of the mouthguard 300 may be configured to space or otherwise isolate the maxillary teeth associated with the upper jaw from the mandibular teeth associated with the lower jaw. As such, the mouthguard may be fabricated from a material that may not be easily punctured by a subject biting it. In some cases, this material may be able to withstand an average bite force of an adult human subject.

Moreover, the base 330 may allow for proper positioning of the mouthguard 300 relative to the mandible or maxilla. For example, the mouthguard 300 may be fabricated from a general shape, that is not customized, therefore the front wall 310 and rear wall 320 may not hold the mouthguard 300 in a set position relative to the teeth of a subject. Therefore, in some embodiments, for example where the mouthguard 300 is placed over the maxillary teeth, a subject may position the base between their mandibular teeth and their maxillary teeth and move their mandible so that the base 330 is surrounded by teeth on at least two sides. The force from the mandible pushing up on the maxillary teeth may hold the mouthguard 300 in a proper position where the maxillary teeth are surrounded by the mouthguard regardless of the orientation of the subject’s head. However, in some embodiments, the general form of the mouthguard may deform around a subject’s teeth to hold the mouthguard in place. Alternatively, or additionally, in some embodiments, when the mouthguard is placed on the maxillary teeth, the base may only contact the mandibular teeth and instead the upward force from the mandibular teeth may be counteracted with the front wall contacting the mucobuccal fold to retain the mouthguard.

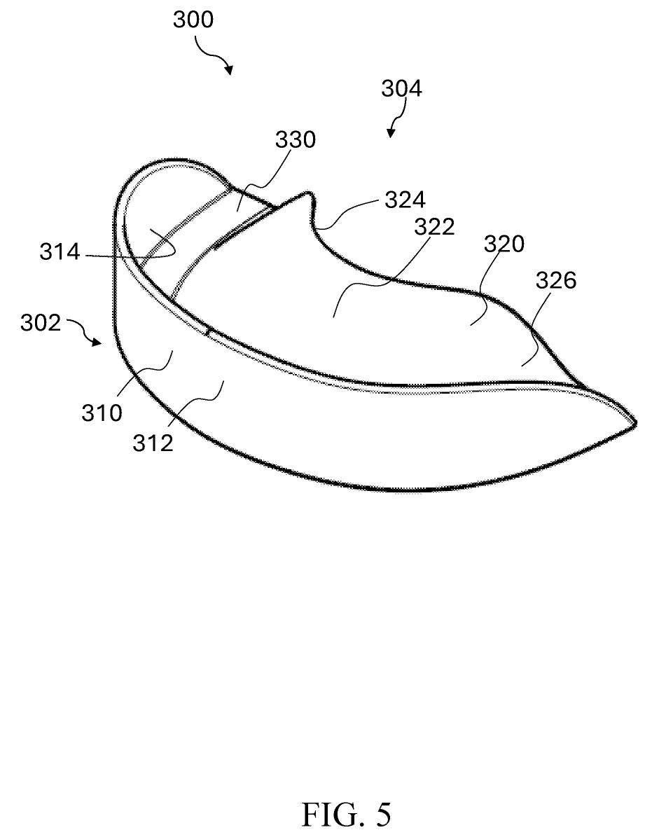

As should be appreciated by the above disclosure, the mouthguard may have any configuration that may isolate the electrically conductive material from a current generated as part of an electricity-based therapy. One such alternative embodiment is detailed in FIG. 5. Like the above-described embodiment, the mouthguard includes a front wall 310, a base 330 and a rear wall 320 that includes a tongue portion 322. In this embodiment, the mouthguard does not extend as posteriorly as the other embodiment. As noted previously, the rear-outer portion 304 of the mouthguard may not be necessary, as the natural anatomy of the facial region may be sufficient to isolate the electrically conductive material from the current. As such, the mouthguard in FIG. 5 may still electrically isolate the non-removable electrically conductive material without contacting all of a subject’s teeth.

Having thus described several aspects of at least one embodiment of this invention, it is to be appreciated various alterations, modifications, and improvements will readily occur to those skilled in the art. Such alterations, modifications, and improvements are intended to be part of this disclosure and are intended to be within the spirit and scope of the invention. Accordingly, the foregoing description and drawings are by way of example only.

What is claimed is:

1. A method of using a mouthguard, comprising:
positioning the mouthguard at least partially over electrically conductive material on or near a subject’s teeth such that the mouthguard is between the electrically conductive material and a target region of the subject;
administering an electricity-based therapy to the target region; and
isolating the electrically conductive material from the electricity-based therapy with the mouthguard by limiting a transmission of a current generated by the electricity-based therapy.

2. The method of claim 1, wherein administering the electricity-based therapy includes generating a microcurrent from an electrical source and placing an electrode in electrical contact with the target region.

3. The method of claim 2, wherein administering the electricity-based therapy includes administering the current at a range of 1 microamperes to 999 microamperes and at a frequency between 0.5 Hz and 100 Hz.

4. The method of claim 1, wherein positioning the mouthguard includes spacing an upper jaw of the subject from a lower jaw of the subject and isolating a tongue from the upper jaw.

5. The method of claim 1, wherein the mouthguard includes a material that prevents or limits the transmission of the current generated by the electricity-based therapy.

6. The method of claim 5, wherein isolating the electrically conductive material includes isolating a buccal mucosa from a non-removable dental device.

7. The method of claim 6, wherein preventing or limiting the transmission of microcurrents includes preventing or limiting the transmission of the current generated from an electrical source to the non-removable dental device.

8. The method of claim 7, wherein the non-removable dental device is at least one of braces, retainers, fillings, dental implants and crowns.

9. The method of claim 8, wherein the target region is associated with skin on the a face of the subject.

10. The method of claim 1, wherein the electricity-based therapy is a cosmetic procedure.

11. The method of claim 1, wherein the electricity-based therapy is a physical therapy procedure.

12. The method of claim 11, wherein the physical therapy procedure is a microcurrent electrical therapy such as a frequency specific microcurrent procedure or microcurrent electrical neuromuscular stimulation therapy.

13. A system for administering an electricity-based therapy comprising:
a device configured to administer the electricity-based therapy to a target region of a subject; and
a mouthguard configured to electrically isolate an electrically conductive material within a mouth of the subject from the electricity-based therapy with the mouthguard by limiting a transmission of a current generated by the electricity-based therapy along and through the mouthguard.

14. The system of claim 13, wherein the mouthguard includes a material that prevents or limits the transmission of the current generated by the electricity-based therapy.

15. The system of claim 14, wherein the device is configured to generate a microcurrent and administer the microcurrent to the subject with an electrode in electrical contact with the target region.

16. The system of claim 14, wherein the mouthguard is configured to electrically isolate a buccal mucosa of the subject from a non-removable dental device.

17. The system of claim 13, wherein the target region is associated with skin on a face of the subject.

18. The system of claim 17, wherein the electrically conductive material within the mouth of the subject is a non-removable dental device and is at least one of braces, retainers, fillings, dental implants and crowns.

19. The system of claim 17, wherein the device is configured to administer a microcurrent electrical therapy such as a frequency specific microcurrent procedure or microcurrent electrical neuromuscular stimulation therapy.

20. The system of claim 13, where in the device is configured to administer the current at a range of 1 microampere to 999 microamperes and at a frequency between 0.5 Hz and 100 Hz.
